(19) United States
(12) Patent Application Publication
(10) Pub. No.: US 2015/0038902 A1
(43) Pub. Date: Feb. 5, 2015

(54) INFUSION THERAPY DEVICE

(71) Applicant: Nico Corporation, Indianapolis, IN (US)

(72) Inventors: Joseph L. MARK, Indianapolis, IN (US); Brian C. DOUGHERTY, Terre Haute, IN (US)

(21) Appl. No.: 14/452,075

(22) Filed: Aug. 5, 2014

Related U.S. Application Data
(60) Provisional application No. 61/862,322, filed on Aug. 5, 2013.

(51) Int. Cl. A61M 25/06

(52) U.S. Cl. CPC A61M 25/0662; A61M 2025/0681; A61M 2025/0687
USPC 604/103

(57) ABSTRACT

An infusion device is disclosed. The infusion device comprises an inner delivery sleeve, an outer sleeve, and a medical balloon. The inner delivery sleeve defines a lumen therethrough, and having at least one opening at or adjacent a distal end that is in communication with the lumen. The outer sleeve is disposed about the inner delivery sleeve. The medical balloon is sealing mounted to the outer sleeve. The outer sleeve includes an inner lumen that is configured to be in communication with a source of pressurized fluid such that the medical balloon may be selectively inflated or deflated. The lumen of the inner delivery sleeve is configured to be in communication with a source of a therapeutic agent such that the therapeutic agent may be selectively delivered through the lumen of the inner delivery sleeve and exit out of the at least one opening, which is positioned distally of the medical balloon.

### CROSS-REFERENCE TO RELATED APPLICATIONS

This application claims priority to U.S. Provisional Patent Application No. 61/862,322, filed Aug. 5, 2013, the disclosure of which is incorporated by reference in its entirety.

### TECHNICAL FIELD

The present disclosure relates generally to a therapeutic device that may be received within a surgical access system for use with delicate and critical tissues.

### BACKGROUND

There exists a need for improved and effective treatment regimens and options for patients, especially for those suffering from brain-related disorders. In certain procedures, once diseased tissue is located within the brain, surgical access systems, such as, for example the surgical access system disclosed in U.S. patent application Ser. No. 13/280,015 provides access to the location of the diseased tissue and a surgical device is used to remove the diseased tissue. Traditionally, once diseased tissue is removed, patients have then been subjected to a “one-size” fits all treatment approach which typically includes a generic and heavy chemotherapy protocol regimen which is delivered systemically. This systemic approach affects the entire body and is designed to provide a balance between enough poison to kill the cancerous cells and tissue without killing all of the healthy tissues. Repeated doses and multiple exposures to radiation are also typically used and delivered by products such as the Gamma Knife and Cyber Knife. However, such treatment regimens are often nothing more than a series of “experiments” on the patient in an effort to find an effective treatment plan. Accordingly, the patient must be monitored to ascertain the effectiveness of the generic therapeutic regimen and continuous modification and tweaking of the treatment regime is performed based upon the positive or negative results of each of the previous successes or failures while attempting to balance sparing healthy tissues and the poisoning effect of the treatment process on the whole patient. Such a treatment regime effectively results in the patient being a guinea pig until a treatment regime is achieved to manage the disease or as in most cases of brain cancers the patient dies from the disease. Unfortunately, in the case of brain cancers, the patient often succumbs to the disease before an effective treatment regime is achieved. Regardless of these heroic clinical efforts that are very biologically caustic to the patient, rarely is any of the current treatment paradigms curative. In fact, since patients diagnosed with brain cancers often do not typically live beyond 9-14 months after initial diagnosis of the disease, long term clinical implications of whole body chemo or target directed radiation therapy are unknown in these patients and may be detrimental if the patient lived long enough for the true impact to be understood.

In addition, most current therapeutic treatment regimens involve delivering immunotherapy or chemotherapy regimens systemically and depend on delivery through the bloodstream. However, the blood-brain barrier, which serves to separate circulating blood from the brain extracellular fluid in the central nervous system (CNS), creates additional challenges to delivering therapeutic agents to specific regions of the brain through the bloodstream. More specifically, the blood-brain barrier actually functions in a neuroprotective role. Thus the blood-brain barrier actually impedes delivery of therapeutic agents to the brain. Therapeutic molecules that might otherwise be effective in therapy are typically larger molecules than the blood brain barrier sieve and for this reason do not cross the blood brain barrier in adequate amounts. In addition to the blood brain barrier, other mechanisms exist within the body to filter out foreign materials and chemicals such as the liver and the kidneys. These filtering actions create additional challenges for the delivery of appropriate concentrations of therapeutics at the intended site of treatment for central nervous system diseases.

To overcome the treatment issues associated with the blood brain barrier, mechanical opening of the blood brain barrier has been proposed, which may complicate the procedure. In addition, use of smaller particles (i.e., nano-particles) have been proposed, whereby the smaller particles are sized to pass through the blood brain barrier, then are attempted to be recombined to form a larger and more effective therapeutic molecule. However, in some instances, the smaller particles fail to recombine in therapeutic levels. Other means to breach the blood brain barrier include delivering chemicals designed to temporarily open up the blood brain barrier to allow for a period of time that larger molecules at therapeutic levels may pass across it. Once across the blood brain barrier, the therapeutic treatment must still get to the diseased tissue, resulting in poisoning healthy tissue, as well as diseased tissue.

Another attempt to breach the blood brain barrier involves using a catheter to try and deliver therapeutic agents in-situ. For example, it has been attempted to place a catheter at a site of interest with therapeutic agents being delivered through an exit arrangement. However, as the therapeutics exit the catheter, (and in some exemplary arrangements, relatively high pressure is used to force the fluid therapeutic agent to exit the catheter) the therapeutics tend to migrate up the track through the tissue, up the outside surface of the catheter. In other words, the therapeutics do not remain at the intended injection site, thereby compromising the desired therapeutic effect to be achieved by the therapeutic agents. Further, as there is no controlled delivery of the therapeutic agent to the intended injection site, a non-homogenous distribution of most therapeutic agents occurs. This unpredictable delivery of therapeutic agent may also compromise effective treatment.

Another attempt that has been employed is convection-enhanced delivery (CED). CED is a method of directly administering drugs into the brain in order to enhance the distribution of drugs throughout the brain parenchyma. CED involves the stereotactic placement through cranial burr holes of catheters into brain parenchyma and the subsequent infusion of therapeutic agents via a microinfusion pump. CED uses a pressure gradient established at the tip of an infusion catheter to push a drug into the extra-cellular space. The intention of this technique is to distribute the drug more evenly, at higher concentrations, and over a larger area than when administered by diffusion alone. However, CED requires the placement of a secondary device that is not integrated to the fluid delivery system. In one instance, the secondary device must be placed in a secondary entry pathway, causing additional trauma to the patient. Alternative approaches involve removing the delivery catheter and then inserting the secondary CED device into the delivery track. If additional therapeutic agent is required during the procedure, then exchange of devices must happen several times during the procedure, lengthening the procedure.

Accordingly, there exists a need for effective treatment regimes that overcomes the challenges created by the blood brain barrier, while efficiently providing targeted treatment to diseased tissue rather than healthy and diseased tissue, with minimal trauma.

### BRIEF DESCRIPTION OF THE DRAWINGS

Exemplary embodiments of the present disclosure will now be described in greater detail with reference to the attached figures, in which:

FIG. 1 is a perspective view of an infusion therapy device;

FIG. 2 is an exploded view of the infusion therapy device of FIG. 1;

FIG. 3 is a perspective view of a cap for the infusion therapy device of FIG. 1; and

FIG. 4 is a perspective view of a hub member for the infusion therapy device of FIG. 1.

### DETAILED DESCRIPTION

Referring now to the discussion that follows and also to the drawings, illustrative approaches to the disclosed instruments and methods are shown in detail. Although the drawings represent some possible approaches, the drawings are not necessarily to scale and certain features may be exaggerated, removed, or partially sectioned to better illustrate and explain the present disclosure. Further, the descriptions set forth herein are not intended to be exhaustive or otherwise limit or restrict the claims to the precise forms and configurations shown in the drawings and disclosed in the following detailed description.

Described herein is an infusion therapy device that is configured to provide for targeted delivery of a therapeutic agent or other desired material to a desired site, while the material is maintained in contact with the targeted site, as well as provide better infiltration of surrounding tissue with the material. In addition, the exemplary infusion therapy device allows for simultaneous insertion of an enhancing infiltration device to further permeation of the therapeutic material without requiring removal of the therapy device.

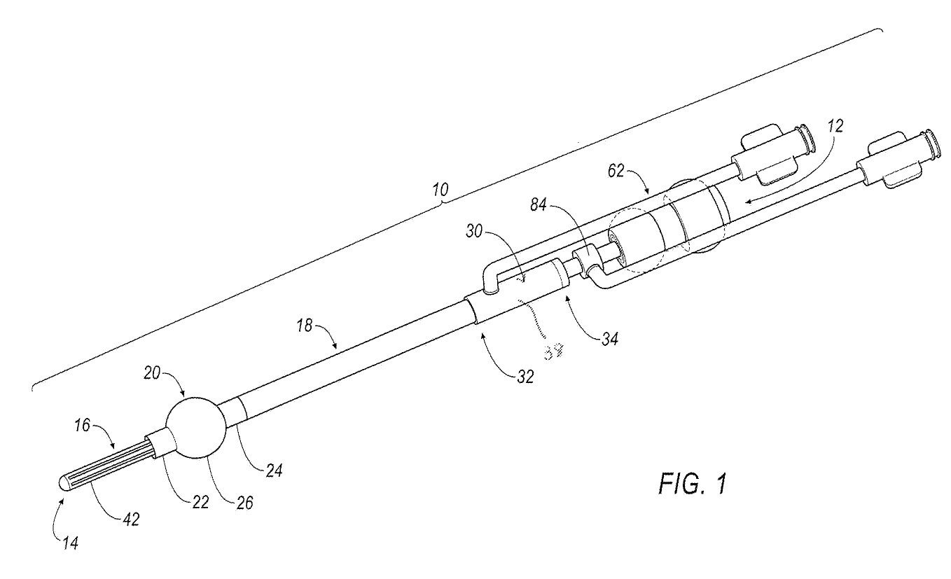

Referring to FIG. 1, an exemplary infusion therapy device 10 is illustrated. Infusion therapy device 10 is defined by a proximal end 12 and a distal end 14 and further includes an inner delivery shaft 16 and outer shaft 18. Outer shaft 18 is slidably disposed over the inner delivery shaft 16. Openings/perforations 42 are disposed through a side wall of the inner delivery shaft 16, adjacent the distal end 14. In one exemplary configuration, openings/perforations 42 may be configured as longitudinal slots. In another configuration, a plurality of circular openings are disposed through the side wall of the inner delivery shaft 16. Openings/perforations 42 are in communication with an inner lumen of the inner delivery shaft 16.

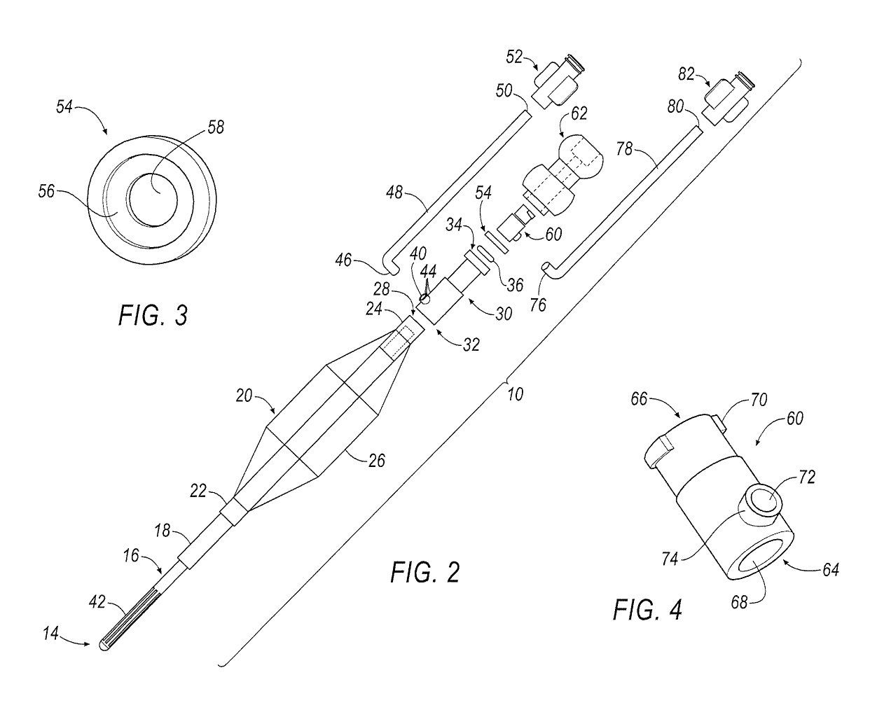

An insufflatable medical balloon 20 is mounted to the shaft 18. Medical balloon 20 is defined by distal and proximal ends 22, 24 with a selectively inflatable body section 26 therebetween. The distal and proximal ends 22, 24 are sealingly engaged to the shaft 18. An opening (not shown) is formed through the sidewall of the outer shaft 18 so as to be in communication with an interior of the medical balloon 20. When inflated, the body section 26 of the medical balloon 20 may be configured to have a spherical shape. Alternatively, the body section 26 may have a central cylindrical shape with tapered sections joining the distal and proximal ends 22, 24, as shown in FIG. 2. Use of the medical balloon 20 will be discussed in further detail below.

Attached to a proximal end 28 of the outer shaft 18 is a first hub 30. First hub 30 includes a first end 32 that is fixed to the proximal end 28 and a second end 34. A body member 38 is disposed between the first and second end 32, 34 and defines a lumen therethrough. A seal member 36 is configured to be disposed within the first hub 30. A delivery opening 40 is in communication with the inner lumen of the body member 38. The seal member 36, which is positioned proximal to the delivery opening 40 serves to direct pressurized fluid or other material entering into the delivery opening 40 into the lumen of the of the outer shaft 18 and into the medical balloon 20 to selectively inflate the medical balloon 20, as will be discussed in further detail below.

Surrounding the delivery opening 40, a mounting flange 44 may be provided. Mounting flange 44 receives a distal end 46 of tubing 48. A proximal end 50 of tubing 48 maybe operatively connected to a luer fitting 52. Fitting 52 may be configured to receive a mating luer fitting (not shown) to direct and maintain a defined amount of pressurized fluid into the medical balloon 20.

A cap member 54 (see FIG. 3) may be frictionally secured to the second end 34 of the first hub 30. The cap member 54 includes an inner depression 56 configured to receive the seal member 36 therein. The cap member 54 serves to prevent the seal member 36 from sliding away from the first hub 30 when the hub 30 (and the connected outer shaft 18) is selectively slid about the inner delivery shaft 16. An opening 58 is configured within the cap member 54 to receive the inner delivery sleeve 16 therethrough.

Referring to FIGS. 2 and 4, a second hub 60 that is configured to be partially disposed within a connector element 62 is shown. In one exemplary arrangement, connector element 62 is a Touhy-Borst connector. Second hub 62 includes a first end 64 and a second end 66. A lumen 68 extends between the first and second ends 64, 66. Adjacent second end 66, locking lugs 70 may be provided that engages with connector element 62. A proximal end (not shown) of the inner delivery shaft 16 is fixedly secured within the second hub 60. The second hub 60 further includes a delivery opening 72 that is in communication with an opening formed through the sidewall of the inner delivery shaft 16 such that the delivery opening 72 is in communication with the lumen of the inner delivery shaft 16.

In alternative arrangement, referring to FIG. 1, a second, smaller hub 84 is shown that is positioned distally of the connector element 62. The alternative hub 84 also includes a first and second end 86, 88 and a lumen 90 therebetween. The alternative hub 84 is sealingly connected to the inner delivery shaft 16 around the opening in the sidewall such that therapeutic agents may be delivered through the inner delivery sleeve 16. A luer fitting (not shown) is spaced from the alternative hub 84 and positioned within the connector element 62.

Surrounding the delivery opening 72, a mounting flange 74 may be provided. Mounting flange 74 receives a distal end 76 of tubing 78. A proximal end 80 of tubing 78 is operatively connected to a luer fitting 82. Fitting 82 may be configured to connect to a source of a therapeutic agent (not shown) and may be used to direct therapeutic agents into the inner delivery sleeve 16. Once therapeutic agents are directed into the inner delivery sleeve 16, the slots 42 permit the therapeutic agents to be directed radially outwardly to the selected site.

The connector element 62 may be selectively opened at the proximal end 12 of the device to allow for an enhancing infiltration device (not shown) to be inserted within the inner delivery sleeve 16. Examples of suitable enhancing infiltration devices include, but are not limited to, convection enhanced delivery devices such as ultrasound, photodynamic wave lengths, electroporation or electropheresis. With this configuration, the enhancing infiltration device may be used simultaneously with the therapeutic agent, without requiring the need to create an additional access track in the patient, and without the need to remove the infusion delivery device 10. Accordingly, trauma to the patient is minimized, as is procedure time.

A method of applying therapy will now be described in connection with the drawings. Before therapy to a specific target site is delivered, access to the site needs to be created. In one exemplary method a surgical access assembly may be employed to create a corridor through which tissue resection and delivery of therapy may be applied. An example of a surgical access assembly and method of using same is described in commonly owned U.S. patent application Ser. No. 13/280,015, the contents of which are incorporated herein by reference in its entirety. The corridor may be created from retracted tissue, or a sheath may be employed to create and maintain the corridor.

Once a corridor has been created, diseased tissue may be removed from the site using a variety of tissue removal devices. One exemplary surgical device that may be used is the surgical cutting device that disclosed in commonly owned U.S. patent application Ser. No. 12/389,447, the contents of which are incorporated by reference in its entirety. The surgical cutting device therein is advantageous in that space is limited to effectuate tissue debulking, such that use of traditional surgical scissors may be challenging, especially if other instruments are inserted into the surgical corridor simultaneously with the surgical cutting device. Moreover, fibrosity of a tumor may present challenges for the use traditional suction debulking devices. And traditional graspers operate by tearing tissue of interest, which may become problematic if vessels or fascicles are too close to the tissue being torn in that such vessels or fascicles may also be torn. Other devices may be used to remove tissue from the area of interest, such as a biopsy instrument.

Once the area of interest for therapy delivery has been prepared, therapy may be delivered to the site. More specifically, the infusion therapy device 10 may be inserted into the created corridor. To prevent therapeutic agents from tracking up the corridor, away from the target area, the outer sleeve 18 may be selectively slid over the inner delivery sleeve 16 so as to position the medical balloon 20 adjacent a target area. Once located at a desired position, the proximal end 24 is occluded and a syringe or other source of pressurized fluid may be engaged to the fitting 52 and actuated to deliver pressurized fluid to the medical balloon 20. This action will inflate the medical balloon 20 against the walls of the surgical corridor, creating a temporary sealed off treatment site.

Next, the fitting 82 is operatively connected to a supply of a suitable therapeutic agent. Once connected, the therapeutic agent is delivered through the inner lumen of the inner delivery sleeve 16, exiting out of the openings/perforations 42. Because the medical balloon 20 is inflated, the therapeutic agent is prevented from tracking up the infusion therapy device within the corridor.

Either while the therapeutic agent is being delivered, or shortly thereafter, an enhancing infiltration device (not shown) is introduced through the proximal end 12 of the infusion device 10 and advanced to the slots 42 disposed at the distal end 14 of the infusion therapy device 10. The enhancing infiltration device is activated, thereby providing improved infiltration of surrounding tissue. Once the therapy has been delivered, the infusion therapy device 10 is removed and any surgical access sheath is removed.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

What is claimed is:

1. An infusion device, comprising:
an inner delivery sleeve defining a lumen therethrough, and having at least one opening at or adjacent a distal end that is in communication with the lumen;
an outer sleeve disposed about the inner delivery sleeve;
a medical balloon sealing mounted to the outer sleeve; wherein the outer sleeve includes an inner lumen and wherein the inner lumen is configured to be in communication with a source of pressurized fluid such that the medical balloon may be selectively inflated or deflated; and
wherein the lumen of the inner delivery sleeve is configured to be in communication with a source of a therapeutic agent such that the therapeutic agent may be selectively delivered through the lumen of the inner delivery sleeve and exit out of the at least one opening.

2. The infusion device of claim 1, wherein the outer sleeve is selectively slidable over the inner delivery sleeve.

3. The infusion device of claim 1, further comprising a connection member mounted to a proximal end of the inner delivery sleeve;
wherein the connection member is configured to provide selective access to the lumen of the inner delivery sleeve.

4. The infusion device of claim 3, wherein the connection member is a Touhy borst connector.

5. The infusion device of claim 1, wherein the at least one opening comprises a series of longitudinally extending slots.

6. The infusion device of claim 1, further comprising a first hub that is fixedly connected to a proximal end of the outer sleeve, wherein the hub includes a delivery opening configured to communicate with a source of pressurized fluid to selectively inflate and deflate the medical balloon.

7. The infusion device of claim 6, further comprising a second hub that is sealingly engaged about a portion of the inner delivery sleeve and in communication with the lumen of the inner delivery sleeve, wherein the second hub includes a delivery opening configured to communication with a source of a therapeutic agent.

8. The infusion device of claim 6, further comprising tubing extending from the opening in the first hub and terminating in a fitting.

9. The infusion device of claim 7, further comprising tubing extending from the opening in the second hub and terminating in a fitting.

10. The infusion device of claim 6, further comprising a seal member disposed around the inner delivery sleeve, and proximal to the delivery opening.

11. The infusion device of claim 10, further comprising a cap member configure to retain the seal member with the first hub.

12. The infusion device of claim 1, further comprising an enhancing infiltration device that is selectively received within the inner delivery sleeve.
